(19) United States
(12) Patent Application Publication
(10) Pub. No.: US 2007/0072226 A1
(43) Pub. Date: Mar. 29, 2007

(54) Mining protein interaction networks

(75) Inventor: Jake Yue Chen, Indianapolis, IN (US)

Correspondence Address: KRIEG DEVAULT LLP, ONE INDIANA SQUARE, SUITE 2800, INDIANAPOLIS, IN, 46204-2079, US

(73) Assignee: Indiana University Research & Technology Corporation

(21) Appl. No.: 11/526,938

(22) Filed: Sep. 26, 2006

Related U.S. Application Data
(60) Provisional application No. 60/721,008, filed on Sep. 27, 2005.

(51) Int. Cl. C12Q 1/68; G06F 19/00

(52) U.S. Cl. 435/6; 702/20

(57) ABSTRACT

One embodiment is a method including creating a protein interaction network including a plurality of protein IDs and a plurality of interactions between protein IDs, determining confidences of interactions of the protein interaction network, identifying a sub-network of the protein interaction network, and determining relevance of proteins of the sub-network to a biological process. Other embodiments include unique systems and methods relating to mining protein interaction networks. Further embodiments, forms, objects, features, advantages, aspects, and benefits shall become apparent from the following descriptions, drawings, and claims.

### CROSS REFERENCE

The present application claims the benefit of U.S. patent application Ser. No. 60/721,008 which was filed Sep. 27, 2005 and is hereby incorporated by reference.

### TECHNICAL FIELD

The technical field relates to identifying, extracting, or mining information from protein interaction networks, and more particularly, but not exclusively, to identifying, extracting, or mining information, such as disease protein biomarkers and drug targets, from protein interaction networks.

### BACKGROUND

Protein interaction networks represent a heretofore unrealized potential to evaluate and characterize the interactions of proteins. Protein interactions are involved in essentially every biological process, including diseases such as Alzheimers Disease, Fanconi Anemia and others, as well as a variety of other biological systems and processes. Present techniques for identifying protein interaction suffer from a number of drawbacks, interactions, and shortcomings including, for example, complexity, inefficiency, inability to characterize protein interaction, false negatives, false positives, and others. There is a need for unique and inventive methods and systems for identifying, extracting, or mining information from protein interaction networks.

### SUMMARY

One embodiment is a method including creating a protein interaction network including a plurality of protein IDs and a plurality of interactions between protein IDs, determining confidences of interactions of the protein interaction network, identifying a sub-network of the protein interaction network, and determining relevance of proteins of the sub-network to a biological process. Other embodiments include unique systems and methods relating to mining protein interaction networks. Further embodiments, forms, objects, features, advantages, aspects, embodiments and benefits shall become apparent from the following descriptions, drawings, and claims.

### BRIEF DESCRIPTION OF THE DRAWINGS

FIG. 1 is a portion of a visualization of a protein interaction network.

FIGS. 2-5 are flowcharts relating to protein interaction network mining methods.

FIG. 6 is a schematic block diagram of a system relating to protein interaction network mining.

FIG. 7 is a schematic diagram of a protein interaction network expansion technique relating to Fanconi Anemia.

FIG. 8 is a visualization of a protein interaction network relating to Fanconi Anemia.

FIG. 9 is a visualization of a protein interaction network relating to Alzheimer Disease.

FIG. 10 is a histogram relating to statistical validation of a protein interaction network relating to Alzheimer Disease.

### DETAILED DESCRIPTION

For the purposes of promoting an understanding of the principles of the invention, reference will now be made to the embodiments illustrated in the drawings and specific language will be used to describe the same. It will nevertheless be understood that no limitation of the scope of the invention is thereby intended, and that all alterations and further modifications of the following embodiments and such further applications of the principles of the invention as would occur to one skilled in the art to which the invention relates are contemplated.

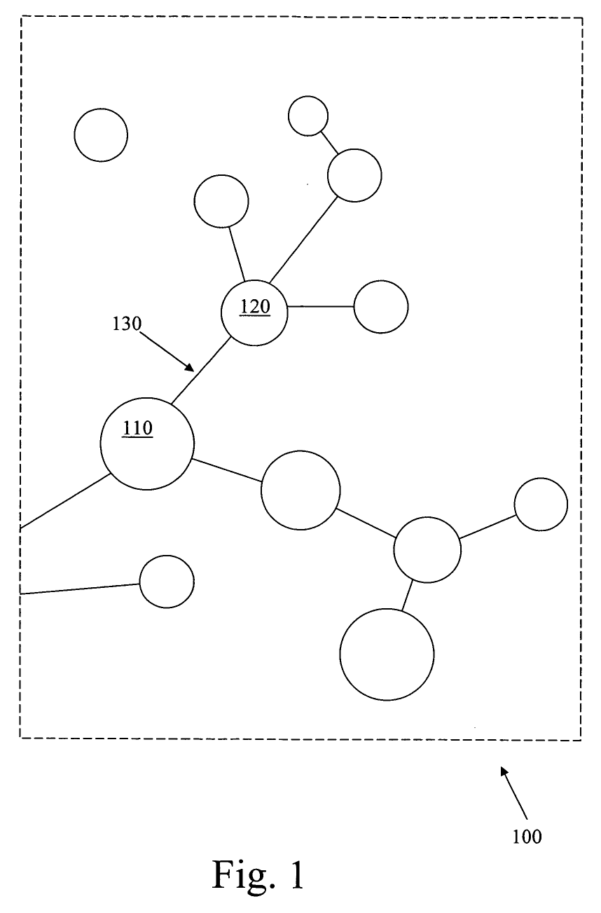

FIG. 1 illustrates one example of a portion of a protein interaction network visualization 100 including a number of nodes, such as nodes 110 and 120, which represent proteins, and a number of lines, such as line 120 which extend between nodes and represent protein interactions. It should be appreciated that visualization 100 is a partial and relatively simple example and that a variety of additional and alternate network visualizations are contemplated. For example, navigable three dimensional network visualization environments could be provided in connection with one or more computers. Additionally, the visualizations could convey a variety of additional information, through color, orientation, size, labeling, animation, length, dashing, shape, thickness, or other characteristics of nodes, lines or other features.

The protein interaction network underlying visualization 100 is one example of a protein interaction network. Protein interaction networks include information regarding direct and/or indirect functional associations of a number of proteins, for example, protein-protein interaction characteristics. Protein interaction networks are typically stored in computer accessible databases, though they could be embodied in essentially any data storage medium or data structure. Protein interaction networks include at least one protein interaction entry, although the database can include a far greater number of entries, for example thousands, millions, or more. Each protein interaction entry includes at least three components: a first ID, a second ID, and an association parameter value. One example of such an entry is: BRAC; ACCA; 0.5. In this example, BRAC is a first protein ID, ACCA is a second protein ID, and 0.5 is an interaction confidence value relating to the first protein ID and the second protein ID. Other exemplary entries can include a variety of additional and/or more particular information, such as binding affinity, equilibrium information such as Keqs, bond strength, bond location, number of bonding sites, toxicity, stability, and virtually any other information regarding interactions between proteins or, more broadly, functional associations between protein and other systems, elements or parameters.

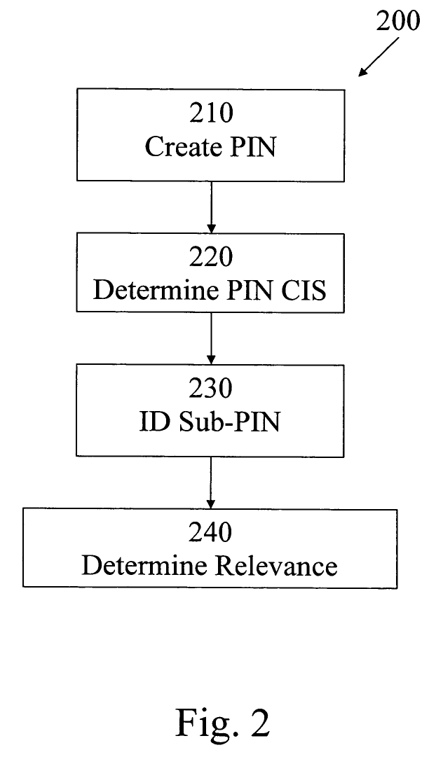

FIG. 2 illustrates a flowchart 200 of one method relating to mining information of a protein interaction network. Flowchart 200 begins at operation 210 where a protein interaction network is created. From operation 210, flowchart 200 proceeds to operation 220 where confidence values for interactions of the protein interaction network are determined. From operation 220, flowchart 200 proceeds to operation 230 where a protein interaction sub-network is identified. From operation 230, flowchart 200 proceeds to operation 240 where relevance of proteins of the protein interaction sub-network to a biological phenomenon, such as a disease, is determined. Thus, flowchart 200 provides one example of determining the relevance of a protein to a biological process. It should be appreciated that the method of flowchart 200 could include a variety of additional, intermediate, or substitute steps including, for example, those herein.

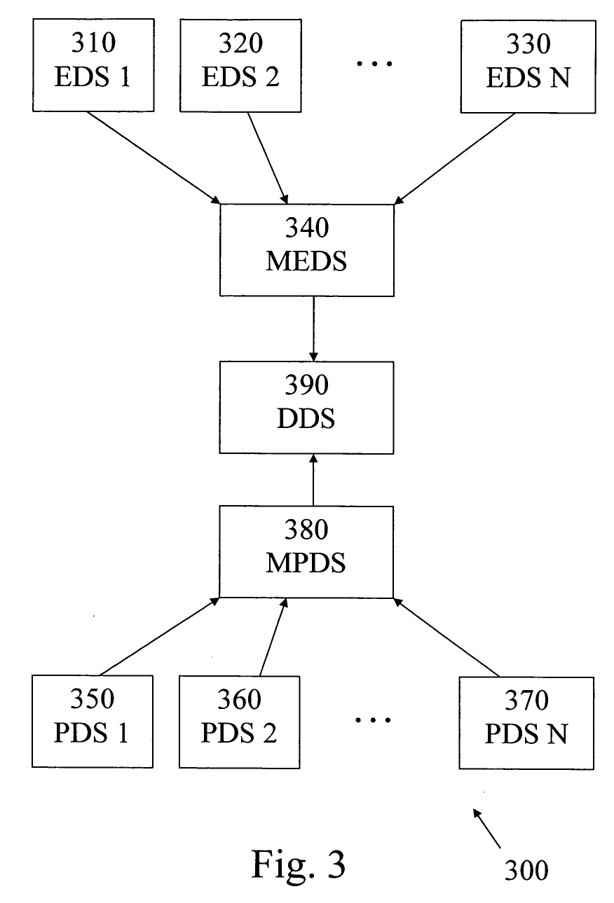

FIG. 3 illustrates a flowchart 300 of another method relating to mining information of a protein interaction network. Flowchart 300 illustrates one example of the creation of a defined data set 390 from which protein interaction information can be mined. The starting constituent components of the defined data set 390 include experimental data sets 310, 320 and 330, and preexisting data sets 350, 360, 370. These data sets could include a variety of data including, for example, the data described herein. As illustrated in FIG. 3, data sets 310, 320, and 330 can be merged, either in series or parallel, at operation 340. Similarly, data sets 350, 360, and 370 can be merged, either in series or parallel, at operation 380. The merged sets 340 and 380 can then themselves be merged to defined data set 390. A variety of other merger operations are also contemplated, for example, successive merger of all constituent data sets into defined data set 390, partial merger of one or more data sets, and still other possible merger or integration operations. Regardless of the particular technique employed, the ultimate product of data set aggregation is ultimately defined by the method of flowchart 300.

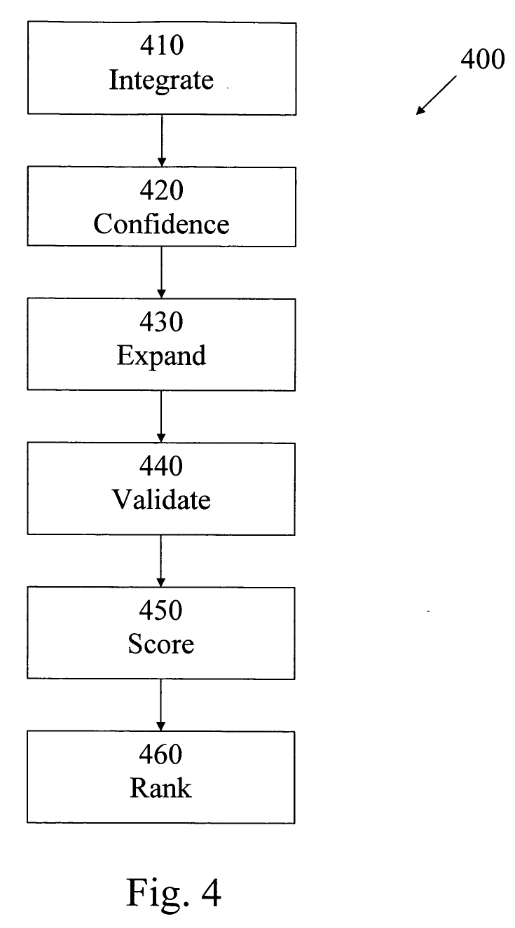

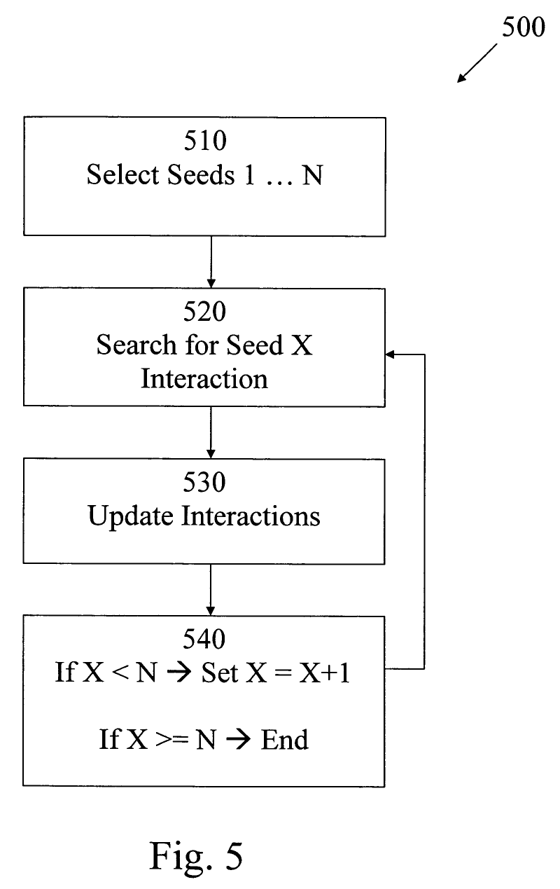

FIG. 4 illustrates a flowchart 400 of a further method relating to mining information of a protein interaction network. Flowchart 400 begins at operation 410. From operation 410 flowchart 400 proceeds to operation 420 where a confidence value is assigned to the interaction, for example, using one or more heuristics or techniques described herein. From operation 420 flowchart 400 proceeds to operation 430 were the protein interaction network is expanded using a technique such as described in connection with FIG. 5 or one or more of the additional network expansion techniques described herein. From operation 430 flowchart 400 proceeds to operation 440 where the expanded network is validated, for example, using the statistical techniques described herein, using network visualization, or using a combination of techniques. From operation 440 flowchart 400 proceeds to operation 450 where proteins of the expanded network are scored according to their relevancy to a biological process, for example, by using a scoring technique such as that described by Equation 1 below. Finally, from operation 450 flowchart 400 proceeds to operation 460 where the scored proteins can be ranked according to their score values.

FIG. 5 illustrates a flowchart 500 of a further method relating to mining information of a protein interaction network. Flowchart 500 begins at operation 510 where one or more seeds are selected. The seed(s) could be genes, expression sequences, proteins, drugs or other molecules which are hypothesized or known to relate to a biological process, such as a disease, cell, tissue, organ, or system, or other target. Furthermore, there are a variety of techniques and resources for selecting the seeds, including microarray experiments, testing a cluster of genes from an expression profile, through genetic, biochemical, or molecular biology and other experiments, by integrating biological databases, through clinical studies, from gene markers, from animal models, or by hypothesis or educated conjecture.

From operation 510 flowchart 500 proceeds to operation 520 where a database such as defined data set 390 mentioned above is searched for interaction with the seed. At this point, additional or all seeds selected above in operation 510 could be searched for interactions, or this could be accomplished iteratively as discussed below. Regardless operation 520 identifies a number of interactions from one or more data sets.

From operation 520 flowchart 500 proceeds to operation 530 where a record of identified interactions is updated. This could be only a single update operation if all seeds were previously checked, or multiple updates could be performed. Regardless, flowchart 500 proceeds to operation 540 which checks whether additional interaction searches or updates should be performed. If so, operations 520 and 530, or just one or the other, are repeated. One example of a logical conditional to test whether further operation should be performed is illustrated in block 540, where the number of seeds checked, X, is checked against the total number of seeds, N, to determine if all seeds have been searched for interactions. Regardless the method of flowchart 500 can produce an expanded interaction network, for example, 10 to 100 times or more.

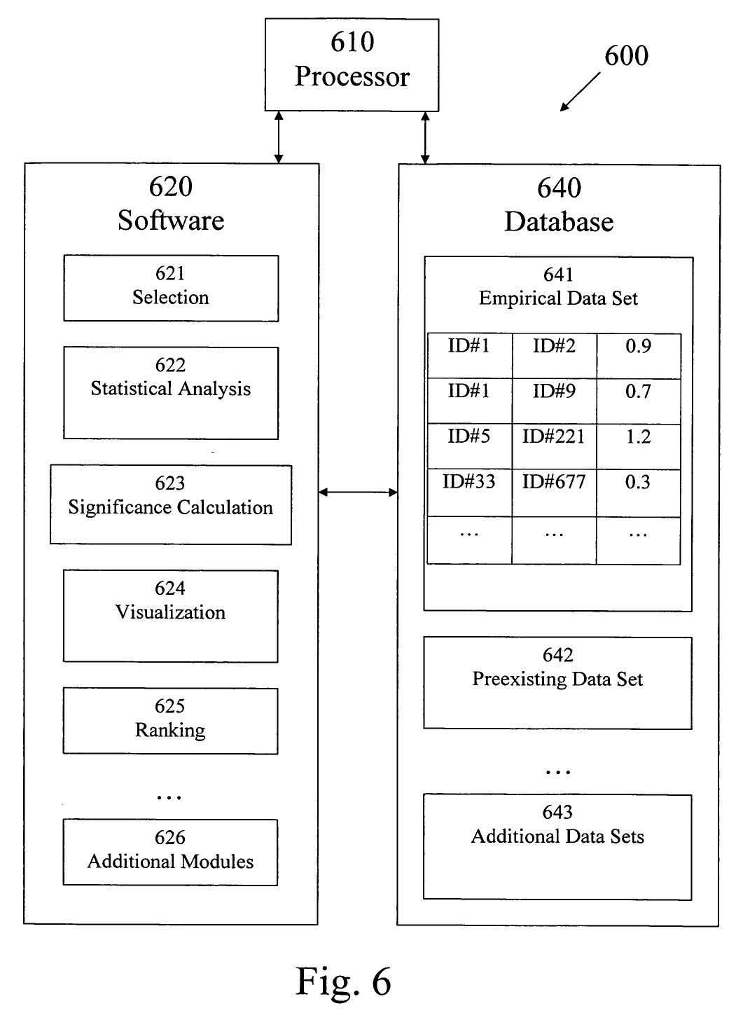

FIG. 6 illustrates a schematic block diagram of a system 600 in which the methods described above, those described below, and others can be implemented. System 600 includes a processor 610, a program environment 620 including one or more programs or program modules, and a database 640 including one or more data sets. Processor 610, program 620 and database 640 are operationally linked as indicated by bi-directional arrows interconnecting them.

Program environment 620 can include a variety of instructions which are executable by processor 610 for selection operations. For example, as illustrated by blocks 621, 622, 623, 624 and 625, the various selection, statistical analysis, significance calculation, visualization, and ranking methods, techniques and operations, including those described above and below, can be performed by processor 610. Also, as indicated by block 626 additional instructions can be carried out by additional modules.

Database 640 includes an empirical data set 641, preexisting data set 642 and can also include additional data sets. The constituent data sets of database 640 can be assembled using the techniques and can include any of the various types of information discussed herein.

The foregoing methods, tools and techniques, as well as others, have been applied in several exemplary data mining operations, one relating to Fanconi Anemia and another relating to Alzheimer Disease, which will now be described.

Fanconi Anemia Data Mining Example

Fanconi Anemia (“FA”) is an autosomal genetic disease with multiple birth defects and severe childhood complications for its patients. The lack of sequence homology of the entire FA complementation group proteins, such as FANCC, FANCG, and FANCA, had made them extremely difficult to characterize. The present example includes a method to extract protein targets for FA, using protein interaction data set collected for FANC group C protein (FANCC). While the method of the present example is described in connection with FA, it applies broadly to other applications disclosed herein.

The present example can be summarized as follows. An initial set of 130 FA interacting proteins, or FANCC seed proteins, was generated by merging an experimentally derived set of FANCC data identified using Tandem Affinity Purification (TAP) pulldown proteomics and data mass spectrometry (MS) techniques with a preexisting human FANCC interacting protein data set. The initial set of FANCC seed proteins was expanded using a nearest-neighbor method to generate a FANCC protein interaction subnetwork of 948 proteins and 903 protein interactions. The subnetwork was evaluated for statistical significances, and indices of aggregation and separations. A visualization of the network was created and examined to confirm that many well connected proteins exist in the network. Ultimately, an interaction network protein scoring algorithm was used to calculate scores indicating the relevance of proteins to FA, and a significance-ranked list of FA proteins was generated.

As mentioned in the summary, the protein interaction data included data from two sources: experimental data, and a preexisting publicly available human protein interaction data set collected through bioinformatics methods. The initial set of FANCC seed proteins was developed based on an initial data set of FA Multi-Protein Complex (MPC) data identified from Tandem Affinity Purification (TAP) protein pulldown and mass spectrometry (MS) experiments. The MPC protein pulldown experiment used protein Fanconi Anemia Complementation Group C (gene symbol: FANCC) as bait, from which a spoke model technique was used to enumerate interacting proteins by counting only the bait-prey protein interactions between FANCC and identified FANCC pulldown proteins. The Online Predicted Human Interaction Database (OPHID) was also searched to retrieve and merge the FANCC MPC data set with preexisting experimental/predicted human interacting protein pairs involving the FANCC protein.

A portion of a FANCC TAP/MS data set showing the 4 of 145 proteins in the MPC having the highest XCorr Scores is shown below in Table 1.

TABLE 1

| Protein | XCorr | Peptide | |
|---|---|---|---|
| ID | Score | Count | Description |
| IPI00023608.1 | 5.585 | 38 | rs\|NP_000127\|sp\|Q00597\|Fanconi_anemia_group_C_protein\|mass\|63429\|Human |
| IPI00296337.2 | 4.994 | 2 | rs\|NP_008835\|sp\|P78527-1\|Splice_isoform1of |
| | | | P78527_DNAdependent_protein_kinase_catalytic_subunit\|mass\|469089\|Human |
| IPI00031801.1 | 4.938 | 1 | rs\|NP_003642\|sp\|P16989-1\|Splice_isoform1of P16989_DNA-binding_protein_A\|mass\|40060\|Human |
| IPI00180305.2 | 4.368 | 1 | rs\|NP_065816\|sp\|\|retinoblastoma-associated factor_600\|mass\|573939\|Human |

The FANCC protein (the first record) served as the bait protein for the proteomics data set. Even though this MPC data gives a list of proteins functionally related to FANCC, the list by itself is not quite informative. In particular, the score, “XCorr Score” is simply a measure of confidence that an entry protein was detected in the MPC proteomics experiment. There are no indicators to forecast how closely and how significantly a protein is related to the FANCC disease biology pathways/networks. The data in the table also showed a nontrivial bioinformatics challenge of making protein identifiers compatible from one data set to another. For example, even though many protein identifiers from public databases are prefixed to the protein description field of each record, the SwissProt ID (immediately following “ . . . |sp|”) in the Protein Description string are missing for proteins “IPI00180305.2”, making it difficult for them to be mapped to protein entries in the SwissProt database.

The second source of data came from the Online Predicted Human Interaction Database (OPHID), a web-based database of human protein interactions with more than 40,000 interactions among approximately 9,000 proteins. It is a comprehensive and integrated repository of known human protein interactions, both from curated literature publications and from high throughput experiments, and of predicted interactions inferred from interaction evidence in model organisms, e.g., yeast, fly, worm, and mouse. Even though more than half of total interactions in OPHID are predicted by mapping interacting protein pairs in available organisms onto orthologous protein pairs in humans, the statistical significance of these predicted human interactions was confirmed by evaluating domain co-occurrence, co-expression, and GO semantic distance evidences.

The entire collection of OPHID data were downloaded and loaded into an Oracle 10G relational database system for analysis. Because there is inherent noise in either MPC proteomics data sets or predicted protein interaction data sets, data reliability was modeled from different data sources. A confidence score was assigned for each protein interaction pair in the merged MPC and predicted human protein interaction data set, based on the following heuristic scoring rules:
MPC protein interactions, of which prey proteins have an “XCorr Score”≧2.5, were assigned a high confidence score of 0.91.
MPC protein interactions, of which prey proteins have an “XCorr Score” between 1.95 and 2.5, were assigned a medium confidence score of 0.75.
OPHID protein interactions that are experimentally collected from humans (non-predicted data set) were assigned a high confidence score of 0.9;
OPHID protein interactions that are inferred from high quality protein interactions in mammalian organisms were assigned a medium confidence score of 0.5;
OPHID protein interactions that are inferred from low quality or low confidence interactions or non-mammalian organisms were assigned a low confidence score of 0.3.

The Human Gene Nomenclature Consortium (HGNC) database, a repository of officially approved gene symbols by an international genome coalition, was also used to resolve protein identifiers from multiple data sources and unofficial gene symbols. The HGNC database provides standard gene symbols and gene mappings to various gene/protein IDs in common public databases such as SwissProt, NCBI RefSeq, NCBI Locuslink, and KEGG enzyme. Using HGNC gene mappings, the majority of protein entries from both the MPC data set and the OPHID database were mapped into SiwssProt IDs and official gene symbols.

As mentioned in the summary, the merged protein interaction data set was expanded with additional OPHID protein interactions. Specifically, expansion of the interaction network was performed on the merged initial protein interaction data set, to derive an FA-related protein interaction sub-network using a nearest-neighbor expansion method which is described as follows.

First, an initial list of FANCC-interacting proteins (merged from both experimental TAP method and OPHID) were denoted as FANCC seed proteins (which include FANCC protein). The set of protein interactions, called FANCC seed interactions, therefore involve FANCC protein as one partner and a seed protein as another partner.

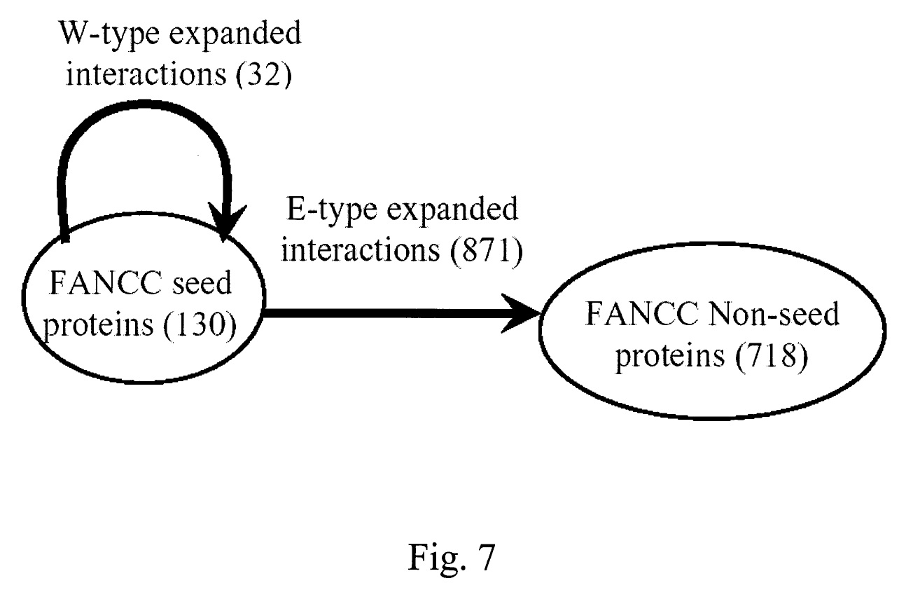

Next, protein interacting pairs in OPHID were searched and retrieved such that at least one member of the protein interaction pair belongs to the FANCC seed proteins. The set of interacting pairs retrieved was called the FANCC expanded interactions, and the new expanded set of proteins was called the FANCC expanded proteins (a superset of FANCC seed proteins). The FANCC expanded interactions had either the “W” type (expansions taking place within seed proteins) or the “A” type (expansions taking place across seed and non-seed proteins). Note that since FANCC-related interactions were not expanded beyond FANCC's immediate interaction partners, interactions with both partners belonging to “non-seed proteins” were not expected. A schematic diagram of this expansion is illustrated in FIG. 7.

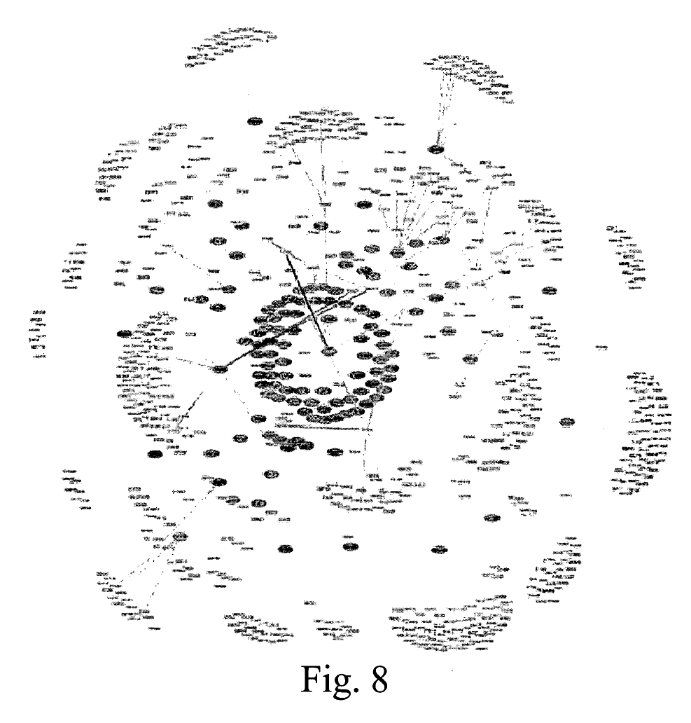

As mentioned in the summary, the merged protein interaction data set was visualized as an FA protein interaction sub-network using interaction confidence and types as parameters. To perform interaction network visualization, a software tool was designed. The tool included native built-in support for relational database access and manipulations. The tool allowed skilled users to browse database schemas and tables, filter and join relational data using SQL queries, and customize data fields to be visualized as graphical annotations in the visualized network. This visualization is illustrated in FIG. 8.

As mentioned in the foregoing summary of the present example, a statistical analysis was used to assess the significance of the information extracted. Since all the FANCC expanded proteins interact with FANCC seed proteins, which in turn interact with the FANCC protein, it was hypothesized that the network formed by the FANCC expanded proteins would be more connected than randomly selected protein sets of the same size. To evaluate network connectivity, several definitions were used. A path between two proteins A and B was defined as a set of proteins P1, P2, . . . , Pn such that A interacts with P1, P1 interacts with P2, . . . , and Pn interacts with B. It was noted that if A directly interacts with B, then the path is the empty set. The largest connected sub-network of a network was then defined as the largest subset of proteins and interactions such that there is at least one path between any pair of proteins in the interaction network subset. The index of aggregation of a network was then defined as the ratio of the size (by protein count) of the largest subnetwork that exists in this network to the size of the network. Therefore, the higher the index of aggregation, the more “connected” the network would be.

The index of separation, a measure of the percentage of W-type interactions found in the entire FANCC expanded interactions was another network gauge used in the present example. It was hypothesized that a high index of separation found in a network represents extensive “re-discovery” of proteins after the protein interactions are expanded from the seed proteins. A simulation method was developed to examine the statistical significance of observed index of aggregation and index of separation in FANCC expanded protein networks. Specifically, the following resampling technique was used to measure how likely an observation was distinctly different from random selections:
Randomly select from OPHID 100 proteins (the number of effectively expandable number of proteins in the FANCC seed protein set).
Build an expanded protein interaction sub-network by using a nearest-neighbor expansion method.
Find the largest connected sub-network and the number of W-type interactions.
Compute the index of aggregation and index of separation for the expanded sub-network.
Repeat above operations 1,000 times to obtain a distribution of the index of aggregation and index of separation under random selection conditions.
Compare the actually observed indexes of aggregation and separation with the distribution obtained in 5 and calculate the p-value.

As mentioned in the summary, a computational technique was used to produce a rank order proteins of high relevance to the FA disease sub-network. The protein target ranking operation evaluated the individual confidence for each protein in the FANCC expanded interaction using a computer implementation of a relevance score function sᵢ for each protein i in the expanded interaction set as is described by Equation 1:

$${s}_{i} = k * ln ( {\sum }_{j ∈ N ( i ) ⋂ A} p ( i , j ) ) - ln ( {\sum }_{j ∈ N ( i ) ⋂ A} N ( i , j ) )$$

where i and j are indices for proteins in the network, k is an empirical constant (k>1), N(i) is the set of interaction partners of protein i in the network, A is the set of FANCC expanded proteins, p(i,j) is the confidence score that was assigned to the interaction between proteins i and j, and N(i,j)=1 if protein j belongs to the intersection of N(i) and A (otherwise N(i,j)=0). To avoid showing a negative score, sᵢ was converted to the exponential scale as tᵢ=exp(sᵢ) and tᵢ as was reported as the final score. The scoring function described by Equation 1 was determined to be favorable in situations in which interacting proteins with many high confidence interactions among its neighbors will stand out among proteins with many low confidence interactions or with only a few interactions.

Mining Alzheimer Disease Data Example

Alzheimer Disease (“AD”) is a progressive neurodegenerative disease with about 4.5 million patients in the U.S. alone. The present example produced a ranking of AD-related proteins based on a set of AD-related genes and a set of human protein interaction data. This example can be summarized as follows. First, an initial seed list of 65 AD-related genes was collected from the Online Mendelian Inheritance in Man (“OMIM”) database and mapped to 70 AD seed proteins. The seed proteins were then expanded to an enriched AD set including 765 proteins using protein interactions from the Online Predicated Human Interaction Database (“OPHID”). It was then verified that the expanded AD-related proteins formed a highly connected and statistically significant protein interaction sub-network. A technique to score and rank-order each protein for its biological relevance to AD pathways(s) was developed and performed. A protein ranking was generated and it was verified that functionally relevant AD proteins were consistently identified by their high ranking. Further details of the present example are as follows.

As mentioned in the summary, the Online Mendelian Inheritance in Man (OMIM) database was searched to obtain an initial set of AD-related genes. The OMIM database includes a number of human gene sequences which include an associated searchable description field. For example, a search was conducted for the term “Alzheimer” which produced 65 OMIM gene records. Regardless of the search term used, the available search capacity suffers from both false positives (containing retrieved genes that are not actually functionally relevant to AD) and false negatives (missing genes that are indeed functionally related to AD but not retrieved), and that the available data does not convey protein interaction information.

The HUGO Gene Nomenclature Committee (HGNC) database was then used to map the initial AD-related genes to AD-related proteins identified by their SwissProt IDs. For each gene in the HGNC database there is a standard gene symbol and gene mappings to various IDs used in common public databases, for example, Swiss-Prot, NCBI RefSeq, NCBI Locuslink, and KEGG enzyme. Initially 65 sets of OMIM gene records, some of which were associated with more than one gene symbol, were selected. After mapping all the gene symbols to protein SwissProt IDs using the HGNC gene mapping table, 70 AD-related proteins were obtained. The slight increase in protein count was due to one-to-many mapping between a gene and its multiple splice variant forms at the protein level.

The Online Predicted Human Interaction Database (OPHID) was also used to collect AD-related protein interaction data. The OPHID database includes more than 40,000 human protein interactions involving 9,000 human proteins, from curated literature publications, high-throughput experiments, as well as predicted interactions inferred from eukaryotic model organisms, such as yeast, worm, fly, and mouse. More than half of OPHID's records are predicted human protein interactions; however, not all OPHID human protein interactions carry the same level of significance, and the problems of both false positives and false negatives are present. To address this concern the present example applied the following heuristic technique to assign a confidence value to the OPHID database: (a) protein interactions from human experimental measurement or from scientific and technical literature were assigned a high confidence score of 0.9; (b) human protein interactions inferred from high-quality interactions in mammalian organisms are assigned a medium confidence score of 0.5; (c) human protein interactions inferred from low quality interactions or non-mammalian organisms are assigned a low confidence score of 0.3.

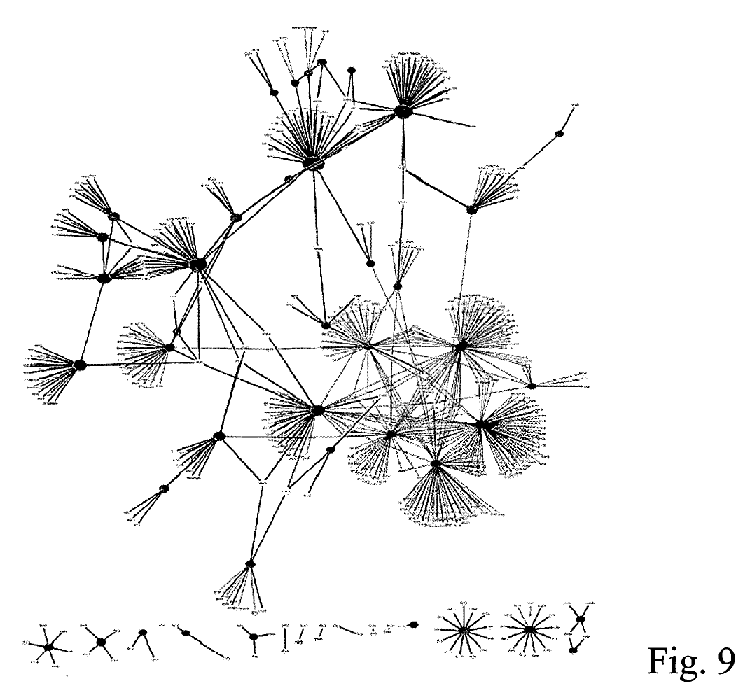

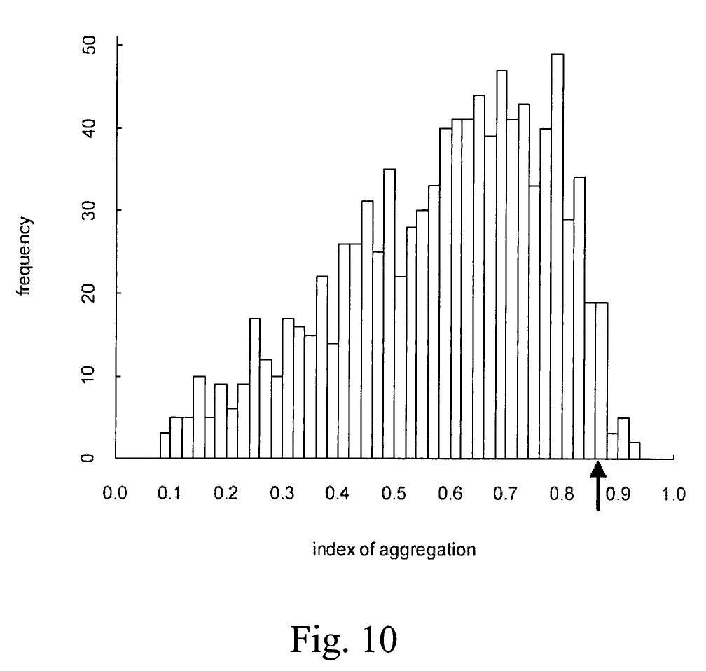

The initial AD-related protein list and OPHID protein interaction data set were then used to derive an AD-related protein interaction sub-network using a nearest-neighbor expansion method. The initial 70 AD-related proteins were selected as the seed-AD-set. To build AD sub-networks, protein interacting pairs in OPHID were pulled out such that at least one member of the pair belongs to the seed-AD-set. This produced an AD-interaction-set. The new set of proteins expanded from initial seed-AD-set by new proteins involved in the AD-interaction-set was identified as the enriched-AD-set (a superset of seed-AD-set). The AD-interaction-set included 775 human protein interactions and the enriched-AD-set contained 657 human proteins identified by Swissprot IDs. The AD protein interaction sub-network was visualized in a manner similar to that described above. A view of the resulting visualization is shown in FIG. 9.

Statistical data analysis tests were conducted to examine the significance of the connected sub-network formed by the AD-interaction-set. It was hypothesized for this statistical evaluation that if the enriched-AD-set indeed identifies functionally related proteins involved in the same process—even if the process were complex and broad—that the connectivity among the enriched-AD-set proteins would be statistically differentiated from that among a set of randomly selected proteins. To formulate this hypothesis precisely, three concepts were used. First, a path between two proteins A and B is defined as a set of proteins P1, P2, . . . , Pn such that A interacts with P1, P1 interacts with P2, . . . , and Pn interacts with B. Note that if A directly interacts with B, then the path is the empty set. Second, the largest connected sub-network of a network was defined as the largest subset of proteins and interactions, among which there is at least one path between any two proteins in the subset. Third, the index of aggregation of a network was defined as the ratio of the size of the largest sub-network that exists in this network to the size of this network. Note that size is calculated as the total number of proteins within a given network/sub-network. To test the hypothesis that the enriched-AD-set proteins are “more connected” than a randomly selected set of protein, a null hypothesis test was developed using the following resampling procedure:
Randomly select from the OPHID database, the same number of human proteins as in the seed-AD-set.
Build the superset of the selected set by using the same nearest-neighbor expansion method described earlier.
Find the largest sub-network of the superset.
Compute the index of aggregation of the superset.
Repeat steps 1 through 4 1,000 times to generate a distribution of the index of aggregation under random selection.
Compare the index of aggregation of the enriched-AD-set with the distribution obtained in 5 and calculate the p-value.

A scoring method was also used to rank proteins in the sub-network, based on their overall roles and contribution to the AD related protein interaction sub-network. The role of a protein in the sub-network can be qualitatively defined as its ability to connect to many protein partners in the network with high specificity (the less promiscuously connected, the better) and high fidelity (the higher the interaction confidence, the better). To define this role quantitatively the relevance score function sᵢ described above in equation 1 was employed. Based on the calculated score functions a protein relevance ranking was generated and output Table 2 shows a portion of the ranking generated:

TABLE 2

| Score | Gene | Description |
|---|---|---|
| 43.01 | APP | amyloid beta (A4) precursor protein |
| | | (protease nexin-II, Alzheimer disease) |
| 36.98 | PSEN1 | presenilin 1 (Alzheimer disease 3) |
| 35.64 | LRP1 | low density lipoprotein-related protein |
| | | 1 (alpha-2-macroglobulin receptor) |
| 21.87 | PSEN2 | presenilin 2 (Alzheimer disease 4) |
| 20.89 | PIN1 | protein (peptidyl-prolyl cis/trans |
| | | isomerase) NIMA-interacting 1 |
| 19.37 | FHL2 | four and a half LIM domains 2 |
| 15.39 | S100B | S100 calcium binding protein, |
| | | beta (neural) |
| 12.96 | FLNB | filamin B, beta (actin binding |
| | | protein 278) |
| 12.37 | CTNND2 | catenin (cadherin-associated protein), |
| | | delta 2 (neural plakophilin-related |
| | | arm-repeat protein) |
| 12.15 | CLU | clusterin (complement lysis inhibitor, |
| | | SP-40,40, sulfated glycoprotein 2, |
| | | testosterone-repressed prostate message |
| | | 2, apolipoprotein J) |
| 11.34 | APBA1 | amyloid beta (A4) precursor protein- |
| | | binding, family A, member 1 (XII) |
| 10.00 | NAP1L1 | nucleosome assembly protein 1-like 1 |
| 9.54 | GTPBP4 | GTP binding protein 4 |
| 9.48 | NCOA6 | nuclear receptor coactivator 6 |
| 9.15 | CDK5 | cyclin-dependent kinase 5 |
| 7.44 | CTSB | cathepsin B |
| 7.29 | ASL | argininosuccinate lyase |
| 4.86 | CTNNB1 | catenin (cadherin-associated protein), |
| | | beta 1, 88 kDa |
| 4.86 | NCKAP1 | NCK-associated protein 1 |
| 4.86 | AGER | advanced glycosylation end product- |
| | | specific receptor |

While the invention has been illustrated and described in detail in the drawings and foregoing description, the same is to be considered as illustrative and not restrictive in character, it being understood that only the preferred embodiments have been shown and described and that all changes and modifications that come within the spirit of the inventions are desired to be protected. It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

What is claimed is:

1. A method comprising:
creating a protein interaction network, the network including a plurality of protein IDs and a plurality of interactions between protein IDs;
determining confidences of interactions of the protein interaction network;
identifying a sub-network of the protein interaction network; and
determining relevance of proteins of the sub-network to a biological process.

2. The method of claim 1 wherein the creating includes combining at least two sets of protein data.

3. The method of claim 1 wherein the creating includes combining experimental protein data with a preexisting protein database.

4. The method of claim 1 wherein the creating includes identifying genes and identifying proteins based upon the identified genes.

5. The method of claim 1 wherein the determining includes applying a heuristic wherein interactions from human experimental measurement are assigned a high confidence, interactions from mammalian organisms are assigned a middle confidence, and interactions from non-mammalian organisms are assigned a low confidence.

6. The method of claim 1 wherein the protein interaction network includes empirically derived interactions and the determining includes separating the empirically derived interactions into at least two confidences.

7. The method of claim 1 wherein the identifying includes utilizing a nearest-neighbor expansion technique.

8. The method of claim 1 wherein the identifying includes defining seed proteins, and selecting interacting pairs including at least one seed protein.

9. The method of claim 1 wherein the determining includes calculating a relevance score function sᵢ for each protein i in the sub-network where
$${s}_{i} = k * ln ( {\sum }_{j ∈ N ( i ) ⋂ A} p ( i , j ) ) - ln ( {\sum }_{j ∈ N ( i ) ⋂ A} N ( i , j ) )$$
where i and j are indices for proteins, k is constant, N(i) is the set of interaction partners of protein i in the network, A is a set of expanded proteins, p(i,j) is the confidence of the interaction between proteins i and j, N(i,j)=1 if protein j belongs to the intersection of N(i) and A, and N(i,j)=0 if protein j does not belong to the intersection of N(i) and A.

10. A method comprising:
integrating at least two data sets to produce an integrated protein interaction data set;
assigning interaction confidence values to the integrated protein interaction data set;
expanding the integrated protein interaction data set to produce an expanded integrated protein interaction data set;
validating the expanded integrated protein interaction data set; and
scoring proteins of the expanded integrated protein interaction data set for relevance to a biological process.

11. The method of claim 10 wherein the validating includes visualizing the expanded integrated protein interaction data set and statistically analyzing the expanded integrated protein interaction data set.

12. The method of claim 10 wherein the validating includes generating a control distribution of indices of aggregation and comparing the expanded integrated protein interaction data set and the control distribution.

13. The method of claim 10 further comprising ranking proteins based upon the scoring proteins of the expanded integrated protein interaction data set for relevance to a biological process wherein the biological process is a disease.

14. The method of claim 10 wherein the scoring includes summing assigned interaction confidence values.

15. A system comprising:
a database including protein association information;
a processor in communication with the database;
a program including instructions executable by the processor to:
select a protein interaction network from the database,
analyze statistical significance of the protein interaction network, and
calculate values indicating significance of proteins of the protein interaction network to a biological process.

16. The system of claim 15 wherein the instructions to select a protein interaction network from the database include instructions to identify a subnetwork.

17. The system of claim 15 wherein the instructions to analyze statistical significance of the protein interaction network include instructions implementing a nearest neighbor expansion method.

18. The system of claim 15 wherein the instructions to calculate values indicating significance of proteins of the protein interaction network to a biological process include instructions to aggregate interaction confidences.

19. The system of claim 15 wherein the program further includes instructions to allow visualization of the protein interaction network.

20. The system of claim 15 wherein the program further includes instructions to rank the calculated values indicating significance of proteins of the protein interaction network to a biological process.
